(19) United States
(12) Patent Application Publication
(10) Pub. No.: US 2017/0259058 A1
(43) Pub. Date: Sep. 14, 2017

(54) Knee brace having electromagnetic stimulators for continual electro-acupunctural stimulation; in vivo and in situ tissue engineering

(71) Applicant: Y. King Liu, Petaluma, CA (US)

(72) Inventor: Y. King Liu, Petaluma, CA (US)

(21) Appl. No.: 15/731,309

(22) Filed: May 22, 2017

Related U.S. Application Data
(63) Continuation-in-part of application No. 13/987,549, filed on Aug. 6, 2013, now Pat. No. 9,687,376.
Application No. 13/987,549 is a continuation-in-part of application No. 13/694,662, filed on Dec. 24, 2012, now Pat. No. 9,629,742.
Application No. 13/694,662 is a continuation-in-part of application No. 12/626,034, filed on Nov. 25, 2009, now Pat. No. 9,440,069.
Application No. 12/626,034 is a continuation of application No. 11/747,075, filed on May 10, 2007, now abandoned.
(60) Provisional application No. 60/799,263, filed on May 10, 2006.

(51) Int. Cl. A61N 1/36; A61N 1/05; A61N 1/32

(52) U.S. Cl. CPC A61N 1/36021; A61N 1/0502; A61N 1/321; A61N 1/36017; A61N 1/326

(57) ABSTRACT

An acupuncture stimulation system comprises a skin overlaying component and a strap having hook-and-loop fastener material, the strap can be wrapped around the skin overlaying component to snugly secure a portion of the system. The skin overlaying component includes: a first electromagnetic form for providing electromagnetic stimulation to a first acupuncture point “Spleen 10”, a second electromagnetic form for providing electromagnetic stimulation to a second acupuncture point “Heting (S 156)”, a third electromagnetic form for providing electromagnetic stimulation to a third acupuncture point “Stomach 34”, a fourth electromagnetic form for providing electromagnetic stimulation to a fourth acupuncture point “Hsiyen (S 145)”, a fifth electromagnetic form for providing electromagnetic stimulation to a fifth acupuncture point “Stomach 35”, and a sixth electromagnetic form for providing electromagnetic stimulation to a sixth acupuncture point “Bladder 40”, to achieve analgesia, cartilage repair and regeneration in the knee joint.

### CROSS-REFERENCE TO RELATED APPLICATIONS

This application claims priority under 35 U.S.C. § 120 as a continuation-in-part application of U.S. patent application Ser. No. 13/987,549, filed on Aug. 6, 2013, which claims priority under 35 U.S.C §120 as a continuation-in-part application of U.S. patent application Ser. No. 13/694,662 filed on Dec. 24, 2012, which claims priority under 35 U.S.C §120 as a continuation-in-part application of U.S. patent application Ser. No. 12/626,034 filed on Nov. 25, 2009, which claims priority under 35 U.S.C. §120 as a continuation application of U.S. patent application Ser. No. 11/747,075 filed on May 10, 2007, which in turn claims priority under 35 U.S.C. §119 to U.S. Provisional Application No. 60/799,263, filed May 10, 2006. The disclosure of all priority applications is hereby incorporated by reference in their entireties.

### FIELD OF THE INVENTION

This invention relates to continual electro-acupunctural stimulation and more specifically to knee braces having electromagnetic stimulators for continual electro-acupunctural stimulation for in vivo and in situ therapeutic effects on analgesia and tissue regeneration and repair.

### BACKGROUND OF THE INVENTION

In China, the insertion of acupuncture needles into acupuncture points to treat diseases has been practiced for at least 2,000 years. The addition of electricity or electro-acupuncture was documented in a French text as early as in 1825. Its author, Chevalier Solardiere, claimed that when static electricity, generated by rubbing a silk scarf against ebony was discharged into the inserted acupuncture needles, the electrical discharge enhanced the therapeutic results of acupuncture. In the 1950's, Chinese acupuncture practitioners used an automobile starter motor as a continuous source of electrical stimuli to acupuncture needles to enhance the efficacy and efficiency of the acupuncture therapeutic effects. Since that original motor-starter stimulator, various electronic stimulators have been employed with a plethora of therapeutic claims. The stimuli delivered by these stimulators to the acupuncture points through the acupuncture needles have varied in frequency, voltage, current, pulse shape and duration. The duration of the electrical stimuli is generally very brief, such as ranging from 10 to 60 minutes. One such system and method can be found in U.S. Pat. No. 7,200,444 to Gavronsky et al., herein incorporated by reference in its entirety.

### BRIEF DESCRIPTION OF THE DRAWINGS

The invention is further illustrated in the following Figures.

FIGS. 1A-1B show an overview of the continual electro-acupuncture stimulation system applied to produce analgesia, repair and/or regeneration of cartilage in the knee joint.

FIG. 2 illustrates a schematic diagram of a knee brace for continual electro-acupuncture stimulation system, according to an embodiment of the invention.

FIG. 3 illustrates a schematic diagram of a knee brace for continual electro-acupuncture stimulation system having a controller unit external to the brace, according to an embodiment of the invention.

FIG. 4 shows a schematic diagram of an electromagnetic form for stimulation, according to an embodiment of the invention.

FIG. 5 shows a knee brace comprising a skin overlaying component and straps having hook-and-loop fastener material, the skin overlaying component includes six electromagnetic forms for stimulation, according to an embodiment of the invention.

FIG. 6 shows schematically dimensions of a 9 mm TO can laser diode, according to an embodiment of the invention.

### DETAILED DESCRIPTION OF THE PREFERRED EMBODIMENT

Disclosed herein are devices and methods for analgesia and tissue repair and regeneration that does not require major surgery or a research-intensive stem cell approach and, at the same time, it is a non-invasive fast-acting treatment. The disclosed treatment is inexpensive when compared to surgical treatments. This recent biomedical breakthrough is a vast improvement over current treatment modalities, whether measured by cost or by time-to-heal effectiveness.

According to some embodiments of the invention, disclosed is a set of electromagnetic stimulators applied externally to certain selected acupuncture points for treating osteoarthritis including pain, joint stiffness, limitation of range of motion and limitation of overall function. The electromagnetic stimulus can be applied to the appropriate anatomical points of the patient such that it is close to the cartilaginous surfaces of an osteoarthritic joint. The electromagnetic stimulation restores the normal electromagnetic field enveloping the joint. For the cartilage tissue, the field stimulates chondrocyte functioning, and increases synthesis of proteoglycans and Type II collagen molecules in cartilage resulting in the efficient and efficacious repair of damaged cartilage. The devices and methods disclosed herein can be more efficient and efficacious than, for example, transcutaneous electrical stimulation through non-specifically placed surface electrodes. Articular cartilage and fibrocartilage repair can potentially take place after three to four weeks of continuous treatment.

Most current treatments for advanced osteoarthritis involve surgically invasive interventions, such as total knee replacement or total hip replacement. These treatments are costly and for some they can be quite perilous in terms of patient morbidity and mortality. Post surgical recovery requires significant time and total joint replacement surgery presents complications or compromised functionality for about 10% of patients. There are few alternative treatments that have shown effectiveness compared to conservative as well as standard surgical practices. Those that do exist require months of treatment. To the contrary, the device and method shown can provide a fast-acting non-invasive alternative treatment that works continuously or substantially continuously and, in most cases, will permit daily patient ambulation, which will further patient rehabilitation.

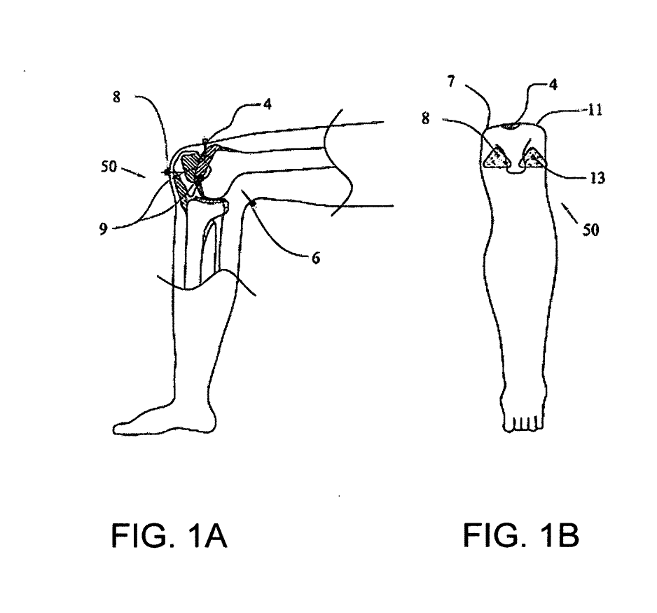

FIGS. 1A-1B illustrates some possible advantageous locations to produce analgesia, repair and/or regeneration of cartilage 9 in the knee joint 50. FIG. 1A is a lateral schematic view of the knee 50 illustrating acupuncture points “Hsiyen (S 145) point”, also known as “eye of the knee” 8, “Heting point (S 156)” 4, and “Bladder 40” 6. FIG. 1B is a frontal schematic view of the knee illustrating other advantageous acupuncture points for knee analgesia, repair, and/or regeneration of cartilage, such as “Hsiyen (S 145)” 8, “Spleen 10” 7, “Heting point (S156)” 4, “Stomach 34” 11, and “Stomach 35” 13. These points can be located by one of ordinarily skill in the art as described below:

“Hsiyen (S 145)” 8: Locate this point with knee flexed, at the lower border of the patella in the depression lateral to the patellar ligament.

“Stomach 34” 11: Locate this point with knee flexed, two finger-widths (comparable to the patients fingers size) above the medio-superior border of the patella on the bulge of the medial portion of the quadriceps femoris muscle.

“Stomach 35” 13: Locate this point in the depression, medial to the patellar ligament, locating the point with the knee flexed.

“Spleen 10” 7: Locate this point with the knee flexed, measure two thumb widths (comparable to the patients thumb size) above the latero-superior border of the patella.

“Heting (S 156)” 4: Locate this point at the depression of the midpoint of the superior patellar border.

“Bladder 40” 6: Locate this point at the midpoint of the transverse crease of the popliteal fossa, between the tendons of the biceps femoris and semitendinosus muscles.

The nature and location of various points and meridians used in Chinese acupuncture are described in many texts, such as the following; the book “Acupuncture in Medical Practice”, Louise O. Wensel, M.D., published 1980 by Reston Publishing (A Prentice Hall Company) is particularly noted; the book “Acupuncture, The Ancient Chinese Art of Healing and How it Works Scientifically”, Felix Mann, M. B. published 1973 by Vintage Books, a division of Random House, New York; the book “Chinese Acupuncture and Moxibustion”, Revised Edition, Chief Editor Cheng Xinnong published 1999 by Foreign Languages Press, Beijing, and; the book “A Manual of Acupuncture” by Peter Deadman et al. published 2001 by Journal of Chinese Medicine Publications. All of these four texts are herein incorporated by reference in their entirety. Appropriate corresponding anatomical landmarks can be selected in order to produce the desired clinical result.

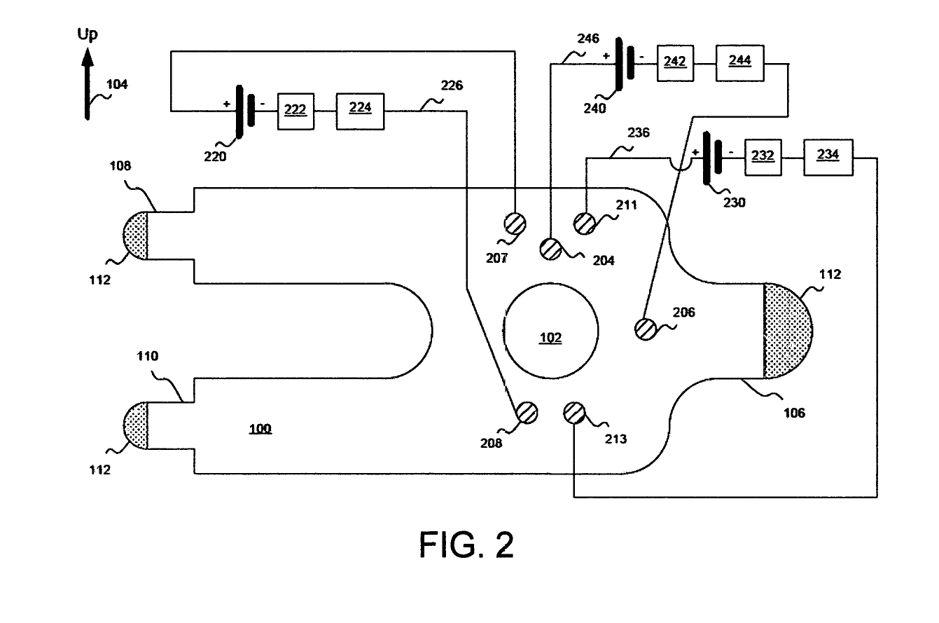

FIG. 2 shows a schematic diagram of a knee brace 100 having three stimulators for continual electro-acupuncture stimulation system, according to an embodiment. Knee brace 100 has a patella-shaped hole 102 for anchoring the knee brace to the knee joint of a patient when knee brace 100 is fixed to the knee. Knee brace is fixed referring to an arrow 104 showing the up direction of the knee. Knee brace 100 comprises non-invasive electrodes 204, 207, 211, 208, 213, and 206.

When knee brace 100 is fixed to the knee: electrode 204 is configured to electrically coupled to acupuncture point “Heting (S 156)” 4; electrode 207 is configured to electrically coupled to acupuncture point “Spleen 10” 7; electrode 211 is configured to electrically coupled to acupuncture point “Stomach 34” 11; electrode 208 is configured to electrically coupled to acupuncture point “Hsiyen (S 145)” 8; electrode 213 is configured to electrically coupled to acupuncture point “Stomach 35” 13; electrode 206 is configured to electrically coupled to acupuncture point “Bladder 40” 6.

In an embodiment, knee brace 100 comprises three individual stimulators 224, 234, and 244 in circuits 226, 236, and 246, respectively, as shown in FIG. 2. Electrodes 207 and 208 are included in circuit 226. Electrodes 211 and 213 are included in circuit 236. Electrodes 204 and 206 are included in circuit 246. The polarity of electrodes 207, 211, and 204 may be positive or negative. Accordingly, the polarity of electrodes 208, 213, and 206 may be negative or positive.

Circuit 226 may include a battery 220 or batteries for supplying electric currents that flow through electrode 207 to electrode 208. Circuit 226 may also include a switch 222 for turning the electric power of battery 220 on and off. The electric current from battery 220 is modulated by an electro stimulator 224. The electric current modulated by electro stimulator 224 is flowing through electrode 207 into the body of a patient to achieve analgesia, cartilage repair and regeneration in the knee joint. The current is then collected from electrode 208 back to circuit 226.

Circuit 236 may include a battery 230 or batteries for supplying electric currents that flow through electrode 211 to electrode 213. Circuit 236 may also include a switch 232 for turning the electric power of battery 230 on and off. The electric current from battery 230 is modulated by an electro stimulator 234. The electric current modulated by electro stimulator 234 is flowing through electrode 211 into the body of the patient to achieve analgesia, cartilage repair and regeneration in the knee joint. The current is then collected from electrode 213 back to circuit 236.

Circuit 246 may include a battery 240 or batteries for supplying electric currents that flow through electrode 204 to electrode 208. Circuit 246 may also include a switch 242 for turning the electric power of battery 240 on and off. The electric current from battery 240 is modulated by an electro stimulator 244. The electric current modulated by electro stimulator 244 is flowing through electrode 204 into the body of the patient to achieve analgesia, cartilage repair and regeneration in the knee joint. The current is then collected from electrode 208 back to circuit 246.

The electrodes may be made of conducting rubber, conducting silicone, or the like, or metal. In an embodiment, closed circuits 226, 236, and 246 may be embedded internally inside the knee brace.

In an embodiment, when knee brace 100 is fixed to the knee, and the electric power is turned on by switch 222, the electric current flows from acupuncture point “Spleen 10” 7 to acupuncture point “Hsiyen (S 145)” 8 in circuit 226. When the electric power is turned on by switch 232, the electric current flows from acupuncture point “Stomach 34” 11 to acupuncture point “Stomach 35” 13 in circuit 236. When the electric power is turned on by switch 242, the electric current flows from acupuncture point “Heting (S 156)” 4 to acupuncture point “Bladder 40” 6 in circuit 246. The currents are inside the body of the patient to achieve analgesia, cartilage repair and regeneration in the knee joint.

Knee brace 100 has at least one strap to fix it to the knee. For example, knee brace 100 may have three straps 106, 108, and 110 to wrap around knee brace 100 for fixing knee brace 100 to the knee. Straps 106, 108, and 110 may have fastening components 112 for fastening knee brace 100. Fastening components 112 may be a hook-and-loop fastener material, such as Velcro.

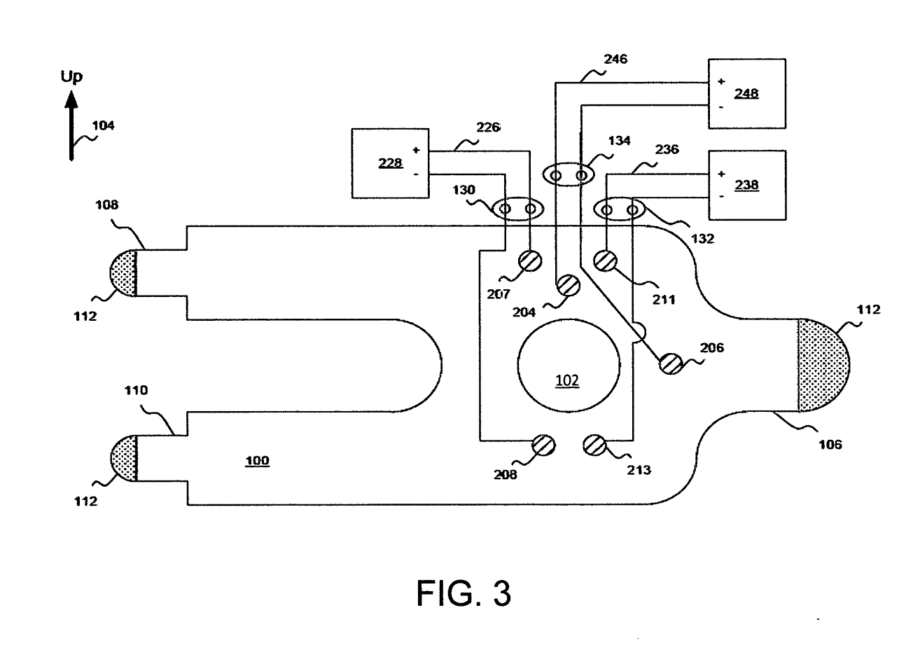

FIG. 3 shows an embodiment where controller units 228, 238, and 248, disposed externally to knee brace 100, according to the disclosure. Controller unit 228 includes battery 220, switch 222, and electro stimulator 224 (not shown). Controller unit 238 includes battery 230, switch 232, and electro stimulator 234 (not shown). Controller unit 248 includes battery 240, switch 242, and electro stimulator 244 (not shown). Other parts of FIG. 3 are the same as FIG. 2. Controller units 228, 238, and 248 may be disposed in a pocket or pockets on knee brace 100.

Controller unit 228 may be connected to circuit 226 through a connector 130. Controller 238 may be connected to circuit 236 through a connector 132. Controller 248 may be connected to circuit 246 through a connector 134. In some implementation, wires connecting to controller units may be long enough for keeping controller units far from knee brace 100, for example, keeping controller units in a pocket or pockets of shirt, dress, or pant worn by the patient. Wires from electrodes to connectors may be embedded in knee brace 100, and only connectors 130, 132, and 134 may be extending from knee brace 100.

One of ordinary skill in the art will readily appreciate that a wide variety of fastening materials can be substituted for hook-and-loop fastener material for any or all of the disclosed components. Such fastening materials include, for example, snap fasteners, button fasteners, adhesives, tapes, buckle fasteners, locks, magnetic fasteners, custom made orthotics, and the like.

In some embodiments, electrical stimulators 224, 234, and 244 can have the following settings: 0-5 milliamp current, 0-18V voltage, 1-100 Hz frequency, 1-99% duty cycle. The pulse waveform is preferably square; however, other morphologies such as triangular, sinusoidal, sawtooth, spike, j-spike, and the like can also be used depending on the desired clinical result. Electrical stimulators 224, 234, and 244 are preferably battery powered; however, they could also be powered by AC outlet using an adaptor (not shown). The battery may be rechargeable. In an embodiment, a square wave of 2 Hz frequency may be applied.

However, it is to be understood that the above construction is only an idealization for ease of illustration, and in reality any of the components could vary in any one or more or any combination of size, shape, size distribution, shape distribution, or other geometric or orthotic characteristics.

In one embodiment, a patient with a disease to be treated, such as osteoarthritis of the knee, is selected. A general overall health assessment for electrotherapy, a focused gait examination, and a Visual Analog Scale (VAS) pain assessment is conducted to better assess the patient's pre-treatment pain. The patient can receive diagnostic bi-planar X-rays and/or Tesler 3 or 7 magnetic resonance imaging (MRI) exams with a patient body weight preload. If the patient is determined to be a suitable candidate, treatment is commenced by fixing the knee brace to patient's knee. In one embodiment, the electrodes at points “Heting (S156)” 4, “Spleen 10” 7, and “Stomach 34” 11 serve as positive electrodes. The electrodes at points “Bladder 40” 6, “Stomach 35” 13, and “Hsiyen (S145)” 8 serve as negative electrodes. In another embodiment, the polarity may be reversed.

The electrodes are operably connected to the electrical stimulator. The power of the stimulator is then increased to a threshold level of sensation by the patient, and then decreased to a sub-sensory level for patient comfort, as well as potentially advantageously promoting analgesia and cartilage repair and/or remodeling. In an embodiment, the practitioner will be able to determine with an appropriate treatment duration depending on the desired clinical result and patient progress through regular serial follow-up visits, physical examinations, pain assessments, radiographs and/or MRIs. In an embodiment, the patient may self-administer the use of the device.

In some embodiments, the electrical stimulator power remains above a threshold sensory level during treatment. In other embodiments, the stimulator power remains sub-sensory throughout the time the electrodes are operably connected to the stimulator. In still other embodiments, the stimulator power can cycle between sensory and sub-sensory power levels during treatment. Candidates for total joint replacement may benefit by undergoing nearly continuous stimulation over an extended period of time. In some embodiments, the system is left in place for 2-5 weeks as a therapeutic trial before contemplating more invasive surgical procedures. In some embodiments, the system can be applied for at least about 1, 2, 3, 5, 7, 10, 14, 21, 28, 35, 42, 60, 90, 120 or more days depending upon the desired clinical result. The various fastening mechanisms disclosed herein can advantageously assist in providing secure implantation of the system for extended periods of time.

The electrical stimulator may be turned on continuously for 24 hours each day. However, in some embodiments, it may be preferable that the electrical stimulator be only activated for only a portion of each day, for example, at least about 1, 2, 3, 4, 6, 8, 10, 12, 14, 16, 18, 20 hours or more each day, and not be active when the patient is more actively moving the area to be treated in activities such as standing, walking, sleeping, or the like. In some embodiments, the electrical stimulator may have a mercury switch automatically that turns off the stimulator when the patient stands and resumes stimulation when the patient is seated or in a recumbent position. In other embodiments, the stimulator can be turned on and off manually.

In an embodiment, knee brace 100 comprises only one of circuits 226, 236, and 246. In another embodiment, knee brace 100 comprises only two of circuits 226, 236, and 246.

In the U.S. patent application Ser. No. 11/747,075 filed on May 10, 2007, which the application claims priority, and which is incorporated by reference in its entirety, the inventor/applicant discloses a method of treating a patient involving the steps of providing a percutaneous acupuncture system that can involve electrical stimulation or other electromagnetic forms for stimulation.

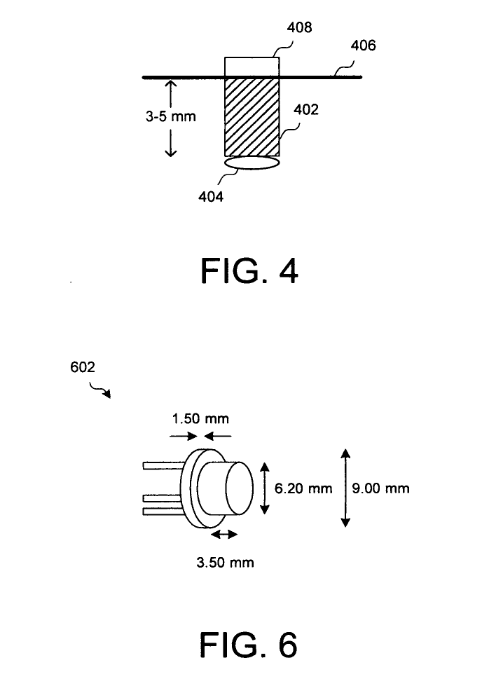

FIG. 4 shows a schematic diagram of an electromagnetic form for stimulation. The electromagnetic form may be an electromagnetic wave 402 provided for stimulating an acupuncture point 404 under skin 406. Acupuncture point 404 may be 3-5 mm under the skin, as disclosed in the U.S. patent application Ser. No. 11/747,075. Electromagnetic wave 402 may be emitted by an electromagnetic source 408. Electromagnetic source 408 may be a laser. The laser may emit a visible electromagnetic wave or an infrared electromagnetic wave.

In the U.S. patent application Ser. No. 11/747,075, it is also disclosed a securable acupuncture system comprising a skin overlaying component having two sides. A first side overlays the skin and a fixator for a needle to provide electrical stimulation. A second side is preferable configured for attachment to another surface. In a preferred embodiment, the second side of the skin overlaying component includes hook-and-loop fastener material, such as Velcro. A strap that preferably has hook-and-loop fastener material on both sides of the strap can be wrapped around the skin overlaying component to snugly secure a portion of the system. The system could be fixed in placed by a custom designed orthotic. For a treatment of knee osteoarthritis, stimulations are given at six acupuncture points around the knee: “Heting (S 156)”, “Spleen 10”, “Stomach 34”, “Bladder 40”, “Stomach 35”, “Hsiyen (S 145)”. Accordingly, the securable acupuncture system comprising the skin overlaying component having hook-and-loop material and the strap having hook-and-loop material to be wrapped around the knee covering six acupuncture points around the knee may be a knee brace, which is a custom designed orthotic.

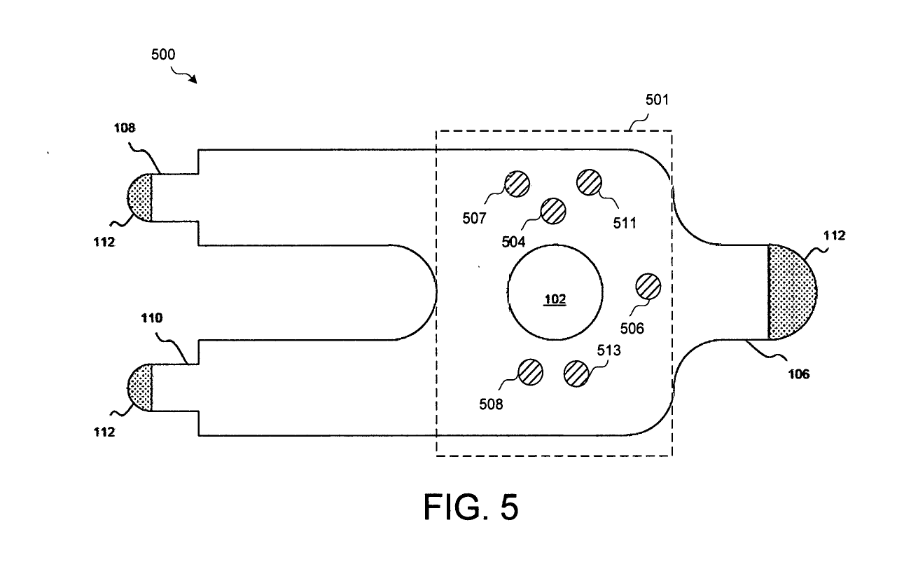

FIG. 5 shows a knee brace 500 comprising a skin overlaying component 501 and straps 106, 108, and 110, having hook-and-loop fastener material 112. Knee brace 500 is similar to knee brace 100 of FIGS. 2 and 3. Straps 106, 108, and 110 are used to wrap around the skin overlaying component 501 to snugly secure a portion of knee brace 500. Skin overlaying component 501 includes six electromagnetic forms 504, 506, 507, 508, 511, and 513 for stimulation.

Electromagnetic form 507 may be an electromagnetic wave configured to stimulate acupuncture point “Spleen 10”. Electromagnetic form 504 may be an electromagnetic wave configured to stimulate acupuncture point “Heting (S 156)”. Electromagnetic form 511 may be an electromagnetic wave configured to stimulate acupuncture point “Stomach 34”. Electromagnetic form 508 may be an electromagnetic wave configured to stimulate acupuncture point “Hsiyen (S 145)”. Electromagnetic form 513 may be an electromagnetic wave configured to stimulate acupuncture point “Stomach 35”. Electromagnetic form 506 may be an electromagnetic wave configured to stimulate acupuncture point “Bladder 40”.

Electromagnetic forms 504, 506, 507, 508, 511, and 513 may be electromagnetic waves emitted by lasers. A laser emitting electromagnetic wave may be a TO (transistor outline) can laser diode 602 as shown in FIG. 6. TO can lasers having diameters 9 mm, 5.6 mm, and 3.8 mm are most commonly available. FIG. 6 shows schematically dimensions of a 9 mm TO can laser diode. Electromagnetic forms 504, 506, 507, 508, 511, and 513 may be emitted by TO can laser diodes. The TO can laser diodes may have each individual power supply, or may have a common power supply. The TO can laser diodes may emit visible electromagnetic waves or infrared electromagnetic waves.

Skin overlaying component 501 may include laser diodes 602 at locations of electromagnetic forms 504, 506, 507, 508, 511, and 513 for emitting electromagnetic waves for stimulating corresponding acupuncture points. Skin overlaying component 501 may further comprises a patella-shaped hole 102 for anchoring knee brace 500 to the knee joint of a patient when knee brace 500 is fixed to the knee.

It is appreciated that the electromagnetic stimulation acupuncture system of FIG. 5 operates in the same way as electro-acupuncture stimulation systems of FIGS. 2 and 3, except TO can laser diodes replacing electrodes.

While the present invention has been described herein with respect to the exemplary embodiments and the best mode for practicing the invention, it will be apparent to one of ordinary skill in the art that many modifications, improvements and sub-combinations of the various embodiments, adaptations and variations can be made to the invention without departing from the spirit and scope thereof For the disclosed methods, the steps need not necessarily be performed sequentially.

The terms used in the following claims should not be construed to limit the invention to the specific embodiments disclosed in the specification and the claims. Rather, the scope is to be determined entirely by the following claims, which are to be construed in accordance with established doctrines of claim interpretation. The present specification and figures are accordingly to be regarded as illustrative rather than restrictive.

What is claimed is:

1. An acupuncture stimulation system, comprising:
a skin overlaying component including:
a first electromagnetic form for providing electromagnetic stimulation to a first acupuncture point “Spleen 10”,
a second electromagnetic form for providing electromagnetic stimulation to a second acupuncture point “Heting (S 156)”,
a third electromagnetic form for providing electromagnetic stimulation to a third acupuncture point “Stomach 34”,
a fourth electromagnetic form for providing electromagnetic stimulation to a fourth acupuncture point “Hsiyen (S 145)”,
a fifth electromagnetic form for providing electromagnetic stimulation to a fifth acupuncture point “Stomach 35”, and
a sixth electromagnetic form for providing electromagnetic stimulation to a sixth acupuncture point “Bladder 40”, to achieve analgesia, cartilage repair and regeneration in the knee joint; and
a strap having hook-and-loop fastener material, wherein the strap can be wrapped around the skin overlaying component to snugly secure a portion of the system.

2. The system of claim 1, wherein each of the electromagnetic forms is an electromagnetic wave emitted by a laser.

3. The system of claim 2, wherein the laser emits one of a visible electromagnetic wave and an infrared electromagnetic wave.

4. The system of claim 2, wherein the laser are a TO can laser diode.

5. The system of claim 4, wherein the skin overlaying component includes:
a first TO can laser diode for emitting an electromagnetic wave for providing electromagnetic stimulation to the first acupuncture point “Spleen 10”,
a second TO can laser diode for emitting an electromagnetic wave for providing electromagnetic stimulation to the second acupuncture point “Heting (S 156)”,
a third TO can laser diode for emitting an electromagnetic wave for providing electromagnetic stimulation to the third acupuncture point “Stomach 34”,
a fourth TO can laser diode for emitting an electromagnetic wave for providing electromagnetic stimulation to the fourth acupuncture point “Hsiyen (S 145)”,
a fifth TO can laser diode for emitting an electromagnetic wave for providing electromagnetic stimulation to the fifth acupuncture point “Stomach 35”, and
a sixth TO can laser diode for emitting an electromagnetic wave for providing electromagnetic stimulation to the sixth acupuncture point “Bladder 40”.

6. The system of claim 5, wherein the TO can laser diodes have individual power supplies.

7. The system of claim 5, wherein the TO can laser diodes have a common power supply.

8. The system of claim 1, wherein the skin overlaying component further comprises a patella shaped hole for anchoring the system to a knee joint of a patient.

9. The system of claim 1, wherein the skin overlaying component and the strap are parts of a knee brace.

10. A method for achieving analgesia, cartilage repair and regeneration in the knee join comprising:
providing a first electromagnetic form for providing electromagnetic stimulation to a first acupuncture point “Spleen 10”,
providing a second electromagnetic form for providing electromagnetic stimulation to a second acupuncture point “Heting (S 156)”,
providing a third electromagnetic form for providing electromagnetic stimulation to a third acupuncture point “Stomach 34”,
providing a fourth electromagnetic form for providing electromagnetic stimulation to a fourth acupuncture point “Hsiyen (S 145)”,
providing a fifth electromagnetic form for providing electromagnetic stimulation to a fifth acupuncture point “Stomach 35”, and
providing a sixth electromagnetic form for providing electromagnetic stimulation to a sixth acupuncture point “Bladder 40”.

11. The method of claim 10 further comprising:
providing a skin overlaying component including the first, second, third, fourth, fifth, and sixth electromagnetic forms; and
providing a strap having hook-and-loop fastener material, wherein the strap can be wrapped around the skin overlaying component to snugly secure the skin overlaying component.

12. The method of claim 11, wherein the skin overlaying component includes:
a first TO can laser diode for emitting an electromagnetic wave for providing electromagnetic stimulation to the first acupuncture point “Spleen 10”,
a second TO can laser diode for emitting an electromagnetic wave for providing electromagnetic stimulation to the second acupuncture point “Heting (S 156)”,
a third TO can laser diode for emitting an electromagnetic wave for providing electromagnetic stimulation to the third acupuncture point “Stomach 34”,
a fourth TO can laser diode for emitting an electromagnetic wave for providing electromagnetic stimulation to the fourth acupuncture point “Hsiyen (S 145)”,
a fifth TO can laser diode for emitting an electromagnetic wave for providing electromagnetic stimulation to the fifth acupuncture point “Stomach 35”, and
a sixth TO can laser diode for emitting an electromagnetic wave for providing electromagnetic stimulation to the sixth acupuncture point “Bladder 40”.

13. The method of claim 12, wherein the TO can laser diodes emit one of a visible electromagnetic wave and an infrared electromagnetic wave.

14. The method of claim 12, wherein the TO can laser diodes have individual power supplies.

15. The method of claim 12, wherein the TO can laser diodes have a common power supply.
